(19) United States
(12) Patent Application Publication
(10) Pub. No.: US 2018/0348208 A1
(43) Pub. Date: Dec. 6, 2018

(54) Systems And Methods For Determining The Risk Of Severe Allergic Reaction To Allergen, And The Immunological Protection Afforded By Vaccines

(71) Applicant: FlowMetric Life Sciences, Doylestown, PA (US)

(72) Inventors: Renold Julius Capocasale, Doylestown, PA (US); Julie Ann Bick, Doylestown, PA (US)

(21) Appl. No.: 15/997,665

(22) Filed: Jun. 4, 2018

Related U.S. Application Data
(60) Provisional application No. 62/514,794, filed on Jun. 3, 2017.

(51) Int. Cl. G01N 33/50; G01N 15/14

(52) U.S. Cl. CPC G01N 33/5091; G01N 2015/1402; G01N 15/14; G01N 33/5047

(57) ABSTRACT

The use of advanced flow cytometry for monitoring allergy sensitivity is mediated by the analysis of the cell mediated immune responses displayed by specialized white blood cells in response to specific allergens.

### RELATED APPLICATIONS

This Application claims priority to and the benefit of U.S. Provisional Application No. 62/514,794, filed on Jun. 3, 2017, the entire teachings of which are incorporated herein by reference.

### FIELD

The present invention relates to methods and systems for determining risk of severe allergic reactions and more particularly to flow cytometry systems and methods for determining allergic reactions.

### BACKGROUND

Vaccines can often be the most effective defense against infectious diseases that can potentially have serious complications or even cause death. The usage of vaccines appears to have prevented possible epidemics and disease outbreaks, and lack of complete vaccine coverage can increase the risk of infection to the entire population, including those who have been vaccinated, because it can reduce the immunity of the entire population.

In addition to vaccine coverage, it appears to be important to ensure vaccine efficacy. Specifically, since vaccines can invoke immune protection by working with an individual's immune system, different patients can respond differently to recommended dosing regimens. Several factors have been shown to influence vaccine efficacy. Such factors can include, for example, a patient's age, sex, genetic background, concurrent medications, previous vaccinations, smoking, diet, psychological stress, infectious diseases, geographical location, socioeconomics/culture, environmental immuno-toxins. Indeed, the kinetics and vigor of an immune response to vaccination can vary significantly.

This type of efficacy variation has most recently been described in the use of the Zoster vaccine, used to reduce the risk of contracting Shingles in those people 50 and over. A recent study demonstrated that individuals with untreated depression displayed significantly less response to the vaccine than those who were either not depressed or who were receiving treatment for the depression. The recommendations from the study were that those patients who displayed this reduced response to Zoster should be transitioned to multi-dose or higher-dose regiments of the vaccine to ensure long-term immunological protection.

Currently, no standardized assay for monitoring or evaluating vaccine efficacy appears to be available. Analysis of vaccine effectiveness generally relies on measuring the levels of circulating antibody titers against the vaccine antigens, a process that only measures B-cell mediated immunity, rather than both B-cell and T cell vaccine memory. Antibody titer measurements often involve a highly the invasive procedure of venous blood draw to acquire one or more vials of blood.

On the other end of the immune spectrum, allergies are typically caused due to the ordinarily protective immune response results in harm to the patient. Allergic reactions can cause a variety of symptoms that range from mild to severe, up to and including anaphylaxis, a serious reaction that is rapid in onset and may cause death. Common allergies include food, insect venom, and drug allergies.

For example, it is estimated that insect stings send more than 500,000 Americans (adults and children) to hospital emergency rooms every year. In addition, it is estimated that food-related anaphylaxis events result in 30,000 emergency room visits per year.

Approximately 8% of children in the United States have a food allergy of some sort and this percentage is on the rise. Eight foods (e.g., egg, milk, peanut, tree nuts, fish, shellfish, wheat, and soy) appear account for 90% of all reactions. Peanuts, tree nuts and shellfish often cause the most severe food allergies. Additionally and significantly, food allergies have been shown to be associated with asthma, and in some cases been described as a trigger for serious immune-related disorders including atopic dermatitis and eosinophilic gastrointestinal disease.

Blood tests utilizing vials of venous blood-draw, or skin prick tests are used to measure allergy sensitivity. Although considered the gold standard of testing for years, the skin prick test can have several significant drawbacks. For example, there is currently no standardization of the test materials or procedure, meaning that the results of the test are dependent on the manufacturer of the test-allergens, the technique of the test administrator, and perhaps most significantly, by the subjective interpretation of the physician evaluating the patient's skin at the end of the test. Additionally, it is also considered somewhat invasive and poorly tolerated, not appropriate for individuals with certain health condition (such as heart failure, certain autoimmune diseases or asthma) and interpretation can be challenging in individuals with dark skin. These factors, coupled with the risk of exposing severely allergic patients to potentially lethal allergens, has generated a real need for a less invasive and safer test (that avoid exposure of individual's to potentially lethal antigens) that can provide increased clarity and value to the diagnosis.

### SUMMARY

The present disclosure is directed, in part, to flow cytometry systems and methods for determining the efficacy and long term immunological protection afforded by vaccination strategies, the safe evaluation of allergen sensitivity, in particular in relation to allergens that may cause anaphylaxis, and to arrays, systems, antibodies, cell-based assays, cell-mediated immune response detection, polynucleotides, and polypeptides, which may be used for practicing such methods.

In one aspect, a method described in which, in a capillary tube comprising sidewalls coated with one or more allergens, a blood sample from a patient is exposed to the sidewalls of the capillary tube to allow interaction of the blood sample with one or more allergens. The blood sample is contacted with a plurality of labeled antibodies that specifically bind to one or more immune cells and one or more immune cell activation markers in the blood sample. The labeled antibodies bound to the one or more immune cells and immune cell activation markers in the blood sample are detected and it is determined whether specific patterns of immune cells in the blood sample are activated by the one or more allergens.

In another aspect, a method for treating a patient by vaccination is described. The method comprises: in a capillary tube comprising sidewalls coated with one or more vaccine antigens, exposing a blood sample from a patient to the sidewalls of the capillary tube to allow interaction of the blood sample with one or more vaccine antigens, contacting the blood sample with a plurality of labeled antibodies that specifically bind to at least one of: one or more T cells, one or more B cells, one or more T cell activation markers, or one or more B cell activation markers in the blood sample, detecting the labeled antibodies bound to the one or more T cells or the one or more B cells in the blood sample, determining whether the one or more T cells or the one or more B cells in the blood sample are activated by the one or more vaccine antigens, and in an event it is determined that one or more of the T cells or B cells are not activated by the one or more vaccine antigens, treating the patient by vaccinating the patient with a vaccine comprising the one or more vaccine antigens or by administering a booster vaccine to the patient with a vaccine comprising the one or more vaccine antigens.

In other examples, any of the aspects above, or any system, method, apparatus described herein can include one or more of the following features.

The blood sample can comprise one or two drops of blood and/or less than 2 milliliter of blood. Additionally or alternatively, the blood sample can comprise between 0.5 milliliter to 3 milliliters of blood.

The blood sample can be allowed to (or brought to) interact with the one or more allergens over a predetermined period of time. The predetermined period of time can be a period of time sufficient to activate specific immune cells in the blood that are responsible for an allergic response. For example, the predetermined period of time can be from about 1 hour to about 24 hours.

The patient can be treated in an event a statistically significant population of the immune cells in the blood are activated by the one or more allergens. The patient can be treated by at least one of: desensitizing the patient to the one or more allergens; counseling the patient to avoid contact with the one or more allergens; or administering to the patient a medicine that reduces allergy symptoms caused by the one or more allergens. Desensitizing the patient to the one or more allergens can be done by administering to the patient an immunotherapy regimen for reducing the patient's allergic response to the one or more allergens.

The specific patterns can be detected using one or more flow cytometry protocols. The one or more allergens can comprise at least one of a food allergen, tree nut allergen, a peanut allergen, a milk allergen, an egg allergen, a shell fish allergen, a drug allergen, an antibiotic allergen, a penicillin allergen, an insect allergen, arachnid venom, bee venom, wasp venom, spider venom.

The one or more immune cells and one or more immune cell activation markers can be basophil identification and basophil activation markers including at least one of: CCR3, CD3, CD13, CD44, CD45, CD54, CD63, CD69, CD71, CD107a, CD107b, CD123, CD164, CD203a, CD203c, CD294, HLA-DR-, and CD300a, or a combination thereof. Additionally or alternatively, the one or more immune cells and one or more immune cell activation markers can be T cell identification and T cell activation markers including at least one of CD25, CD26, CD27, CD28, CD30, CD4L, CD71, CD95, CD134, CD154, and HLA-DR, or a combination thereof. In some aspects, the one or more immune cells and one or more immune cell activation markers can be dendritic cell identification and dendritic cell activation markers including at least one of CD1a, CD1b, CD40, CD80, CD86, CD128b, and CD209, or a combination thereof. Additionally or alternatively, the one or more immune cells and one or more immune cell activation markers can be mast cell identification and mast cell activation markers including at least one of CD2, CD25, CD35, CD88, CD203a, and CD117, or a combination thereof.

In some aspects, the one or more immune cells and one or more immune cell activation markers can be eosinophil identification and eosinophil activation markers including at least one of CD15, CD23, CD52, CD53, CD88, CD129, CD69+, CD62L (decrease), CCR3, CCR9, CCR4, and CXCR3, or a combination thereof. Additionally or alternatively, the one or more immune cells and one or more immune cell activation markers can be monocyte identification and monocyte activation markers including at least one of CD14, CD45, CD86, CD16, CD69, TNFα, CCR5, CD38, CX3CR1, CD11b, and HLA-DR, or a combination thereof.

In some embodiments, the blood can be contacted with detectably-labeled antibodies that specifically bind CD3, CD45, CD69, CD71, CD203c, and CD294. Further, the labeled antibodies bound to one or more of basophils, T cells, dendritic cells, mast cells, eosinophils, or monocyte cells can be detected using a flow cytometer.

Further, the present disclosure further provides methods for treating allergy sensitivity in a patient, comprising: a) inserting a sample of capillary blood from the patient into a tube or collection chamber comprising sidewalls coated with one or more allergens, and allowing the blood to interact with the one or more allergens for a period of time sufficient to activate specific immune cells in the blood that are responsible for an allergic response; b) after or during the period to allow the blood to interact with the one or more allergens according to step a), contacting the blood with a plurality of detectably-labeled antibodies that specifically bind to one or more immune cells and one or more immune cell activation markers; c) detecting the labeled antibodies bound to the one or more immune cells and immune cell activation markers to determine whether specific patterns of immune cells in the blood were activated by the one or more allergens; and d) treating the patient comprising: i) desensitizing the patient to the one or more allergens; ii) counseling the patient to avoid contact with the one or more allergens; and/or iii) administering to the patient a medicine that reduces the allergy symptoms; if it is determined that a statistically significant population of immune cells were activated by the one or more allergens.

The present disclosure also provides methods for treating a patient by vaccination comprising: a) inserting a sample of capillary blood from the patient into a tube or collection chamber comprising sidewalls coated with one or more vaccine antigens, and allowing the blood to interact with the one or more antigens for a period of time sufficient to activate specific immune cells in the blood; b) during or after allowing the blood to interact with the one or more vaccine antigens according to step a), contacting the blood with a plurality of detectably-labeled antibodies that specifically bind to one or more T cell and/or B cell identification markers and/or one or more T cell and/or B cell activation markers; c) detecting the specifically labeled antibodies bound to the one or more of the T cells and/or B cells to determine whether one or more of the T cells and/or B cells in the blood were activated by the vaccine antigen; and d) treating the patient by vaccinating the patient with a vaccine comprising the one or more vaccine antigens or by administering a booster vaccine to the patient with a vaccine comprising the one or more vaccine antigens; if it is determined that one or more of the T cells and/or B cells were not activated by the one or more vaccine antigens.

The present disclosure also provides methods for treating allergy sensitivity in a patient in need thereof, comprising: a) requesting a test providing the results of an analysis of a sample of blood from the patient for the presence of a statistically significant population of one or more immune cells activated by one or more allergens determined by contacting the blood with a plurality of detectably-labeled antibodies that specifically bind to: i) one or more basophil identification and basophil activation markers selected from the group consisting of CCR3, CD3, CD13, CD44, CD45, CD54, CD63, CD69, CD71, CD107a, CD107b, CD123, CD164, CD203a, CD203c, CD294, HLA-DR-, and CD300a, or any combination thereof; ii) one or more basophil identification and basophil activation markers selected from the group consisting of CD3, CD13, CD45, CD63, CD69, CD71, CD107a, CD164, CD203c, and CD294, or any combination thereof; iii) one or more T cell identification and T-cell activation markers selected from the group consisting of CD25, CD26, CD27, CD28, CD30, CD40L, CD71, CD95, CD134, CD154, and HLA-DR, or any combination thereof; iv) one or more dendritic cell identification and dendritic cell activation markers selected from the group consisting of CD1a, CD1b, CD40, CD80, CD86, CD128b, and CD209, or any combination thereof; v) one or more mast cell identification and mast cell activation markers selected from the group consisting of CD2, CD25, CD35, CD88, CD203a, and CD117, or any combination thereof; vi) one or more eosinophil identification and eosinophil activation markers selected from the group consisting of CD15, CD23, CD52, CD53, CD88, CD129, CD69+, CD62L (decrease), CCR3, CCR9, CCR4, and CXCR3, or any combination thereof; vii) one or more monocyte identification and monocyte activation markers selected from the group consisting of CD14, CD45, CD86, CD16, CD69, TNFα, CCR5, CD38, CX3CR1, CD11b, and HLA-DR, or any combination thereof; and/or viii) one or more immune cell markers selected from the group consisting of CD3, CD45, CD69, CD71, CD203c, and CD294; wherein the patient is diagnosed with an allergy sensitivity if a statistically significant population of activated basophils, T cells, dendritic cells, mast cells, eosinophils, and/or monocyte cells are detected in the blood; and b) treating the patient comprising: i) desensitizing the patient to the one or more allergens; ii) counseling the patient to avoid contact with the one or more allergens; and/or iii) administering to the patient a medicine that reduces the allergy symptoms.

The present disclosure also provides methods for treating allergy sensitivity in a patient in need thereof, comprising: a) analyzing a blood sample from the patient for the presence of a statistically significant population of immune cells activated by one or more allergens compared to blood from a healthy patient; and b) treating the patient by: i) desensitizing the patient to the one or more allergens; ii) counseling the patient to avoid contact with the one or more allergens; and/or iii) administering to the patient a medicine that reduces the allergy symptoms; wherein the presence of activated immune cells is determined by contacting the blood with a plurality of detectably-labeled antibodies that specifically bind to: i) one or more basophil identification and basophil activation markers selected from the group consisting of CCR3, CD3, CD13, CD44, CD45, CD54, CD63, CD69, CD71, CD107a, CD107b, CD123, CD164, CD203a, CD203c, CD294, HLA-DR-, and CD300a, or any combination thereof; ii) one or more basophil identification and basophil activation markers selected from the group consisting of CD3, CD13, CD45, CD63, CD69, CD71, CD107a, CD164, CD203c, and CD294, or any combination thereof; iii) one or more T cell identification and T-cell activation markers selected from the group consisting of CD25, CD26, CD27, CD28, CD30, CD40L, CD71, CD95, CD134, CD154, and HLA-DR, or any combination thereof; iv) one or more dendritic cell identification and dendritic cell activation markers selected from the group consisting of CD1a, CD1b, CD40, CD80, CD86, CD128b, and CD209, or any combination thereof; v) one or more mast cell identification and mast cell activation markers selected from the group consisting of CD2, CD25, CD35, CD88, CD203a, and CD117, or any combination thereof; vi) one or more eosinophil identification and eosinophil activation markers selected from the group consisting of CD15, CD23, CD52, CD53, CD88, CD129, CD69+, CD62L (decrease), CCR3, CCR9, CCR4, and CXCR3, or any combination thereof; vii) one or more monocyte identification and monocyte activation markers selected from the group consisting of CD14, CD45, CD86, CD16, CD69, TNFα, CCR5, CD38, CX3CR1, CD11b, and HLA-DR, or any combination thereof; and/or viii) one or more immune cell markers selected from the group consisting of CD3, CD45, CD69, CD71, CD203c, and CD294.

Other aspects and advantages of the invention can become apparent from the following drawings and description, all of which illustrate the principles of the invention, by way of example only.

### BRIEF DESCRIPTION OF THE DRAWINGS

Features and advantages of the invention described herein, together with further advantages, may be better understood by referring to the following description taken in conjunction with the accompanying drawings. The drawings are not necessarily to scale, emphasis instead is generally placed upon illustrating the principles of the invention. The dimensions of the various features are arbitrarily expanded or reduced for clarity.

FIG. 1 illustrates flow cytometry plots showing staining for activation markers on unstimulated T cells.

FIG. 2 depicts flow cytometry plots illustrating staining for activation markers on T cells stimulated with 10 μg/mL of Phytohemagglutinin (PHA), a non-specific activator of the cell mediated immune response.

FIG. 3 depicts flow cytometry plots illustrating staining for activation markers on T cells stimulated with 20 μg/mL of PHA.

FIG. 4 illustrates flow cytometry plots depicting staining for activation markers on T cells stimulated with 30 μg/mL of PHA.

FIG. 5 illustrates activation of FoxP3+/CD25+ upon CD4+ T cells stimulated with increasing concentrations (0, 20, 40, 60, 80, 100 μg/mL) of PHA.

FIG. 6A illustrates the percentage of FoxP3-positive CD4+ T cells from the blood of three individual donors stimulated with increasing concentrations (0, 20, 40, 60, 80, 100 μg/mL) of PHA.

FIG. 6B illustrates the average percentage of FoxP3-positive CD4+ T cells from the donor pool, as stimulated with increasing concentrations (0, 20, 40, 60, 80, 100 μg/mL) of PHA.

FIG. 7 illustrates activation of CD71+ T cells stimulated with increasing concentrations (0, 20, 40, 60, 80, 100 μg/mL) of PHA.

FIG. 8A illustrates the percentage of CD71-positive CD4+ T cells from the blood of three individual donors stimulated with increasing concentrations (0, 20, 40, 60, 80, 100 μg/mL) of PHA.

FIG. 8B illustrates the average percentage of CD71-positive CD4+ T cells from the donor pool, as stimulated with increasing concentrations (0, 20, 40, 60, 80, 100 μg/mL) of PHA.

FIG. 9 illustrates activation of CD69+ T cells stimulated with 100 μg/mL.

FIG. 10A illustrates the mean fluorescence intensity of CD69 staining in T cells from the donor pool as simulated with increasing concentrations (0, 20, 40, 60, 80, 100 μg/mL) of PHA.

FIG. 10B illustrates the average percentage of CD69-positive CD4+ T cells from the donor pool, as stimulated with increasing concentrations (0, 20, 40, 60, 80, 100 μg/mL) of PHA.

FIGS. 11A, 11B, 11C, and 11D illustrate the flow cytometry gating (analysis) strategy used to identify cell population responses indicative of a severe allergic response.

FIG. 11A illustrates an unstimulated control sample.

FIG. 11B illustrates a control sample stimulated by activation with fMLP.

FIG. 11C illustrates an example of an allergic reaction.

FIG. 11D illustrates a control sample collected from an individual without a reported allergy to Amoxicillin.

### DETAILED DESCRIPTION

The present disclosure provides a non-invasive system by which the immunological response of individual patients to FDA-approved vaccine regimens can be comprehensively evaluated. These vaccination regimens (vaccine dose, boosting frequency or vaccine formulation) can be modified to help ensure appropriate immunological protection of that individual is achieved.

Flow cytometry can allow scientists to examine individual cells. These cells, such as white blood cells, can be labeled using antibodies conjugated with fluorescent molecules. Each antibody species binds with a specific protein that can be present on the surface or in the cytoplasm of cells. Flow cytometry can measure the presence or absence of the fluorescently-labeled antibody on or in cells, indicating the presence or absence of the target cellular protein, by passing the cells in a uniform stream through a laser beam. The emission spectra from the fluorescently labeled antibodies can be detected by photomultiplier tube detectors.

By using a combination of antibody panels, coupled with optimized fluorescent molecules, it is possible to detect and enumerate rare protein activation markers indicative of cellular events in a biological sample, such as cell mediated immune responses. The detection of a cell mediated immune response in response to the presence of specific antigens can be utilized in vitro to diagnose allergen sensitivity and vaccine efficacy within individuals. Particular cell surface markers can be used to identify linage of cells, while others can be used to determine activation or a specific event within those cell types. It is the combination of the lineage and activation markers that can be used in determining a clinical diagnosis or monitoring prognosis.

In some aspects, embodiments disclosed herein can evaluate of an individual's sensitivity to specific allergens, in particular those allergens associated with anaphylaxis in a given population, determine the level of immunological protection afforded to an individual by routine vaccination regimens, and allow the use of modified vaccine dosing, vaccine formulation or boosting regimens to ensure enhanced protection of that individual. For example, in some aspects, highly sensitive and robust assays are provided to analyze an individual's cell mediated immune response through the detection of cell activation markers expressed on specific cell types of the immune system. The expression of these activation markers in vitro can be stimulated through the exposure of an individual's white blood cells to specific antigens.

For allergy sensitivity evaluation, these antigens or allergens can be proteins that result in allergy responses in sensitive individuals, through explicit activation of specific cells of the immune system. Using small volumes of capillary blood (about 0.1 to 0.2 mL), fluorescently labeled antibodies, specific to the identification and activation markers for immune responsive cells can be used to label specifically allergen-responsive activated cells. These cells can be detected through advanced flow cytometry protocols that utilize absolute cell counts for the detection of even rare cellular activation events.

In regard to the vaccine efficacy evaluation, small volumes of capillary blood (e.g., about 0.1 to 0.2 mL) can be used to measure the vaccine-antigen dependent activation of memory and regulatory immune cells. The profile of the observed vaccine-antigen dependent cellular response can be directly related to the long-term immunological protection afforded to that individual and can, therefore, provide information relating to the efficacy of vaccine regimens used to treat that individual in terms of immunological protection.

In some aspects, embodiments disclosed herein can relate to treating allergy sensitivity in a patient. Specifically, in some aspects, a sample of blood from the patient is collected into a capillary tube or capillary draw device chamber that comprises sidewalls coated with one or more allergens. The collected blood is allowed to interact with the one or more allergens for a period of time sufficient to activate specific immune cells in the blood that are responsible for an allergic response. The time period can be any suitable time period, for example from 1 hour to 24 hour. After or during the period to allow the blood to interact with the one or more allergens, the blood is brought to contact with a plurality of detectably-labeled antibodies that specifically bind to one or more immune cells and one or more immune cell activation markers. Labeled antibodies bound to the one or more immune cells and immune cell activation markers are detected and utilized to determine whether specific patterns of immune cells in the blood were activated by the one or more allergens. The patient can be treated by: i) desensitizing the patient to the one or more allergens; ii) counseling the patient to avoid contact with the one or more allergens; and/or iii) administering to the patient a medicine that reduces the allergy symptoms, if it is determined that a statistically significant population of immune cells were activated by the one or more allergens.

The allergen can be a food allergen, such as a tree nut allergen or a peanut allergen. Alternatively or additionally, the food allergen can be a milk allergen, egg allergen, or shell fish allergen. In some embodiments, the allergen is a drug allergen, such as an antibiotic allergen (e.g., a penicillin allergen). Additionally or alternatively, the allergen can be an insect or arachnid venom (e.g., spider venom). For example, the insect venom can be a bee or wasp venom.

In some embodiments, the one or more immune cells and one or more immune cell activation markers can be basophil identification and basophil activation markers, such as CCR3, CD3, CD13, CD44, CD45, CD54, CD63, CD69, CD71, CD107a, CD107b, CD123, CD164, CD203a, CD203c, CD294, HLA-DR-, and CD300a, or any combination thereof. Additionally or alternatively, the basophil identification and basophil activation markers can be including any of CD3, CD13, CD45, CD63, CD69, CD71, CD107a, CD164, CD203c, and CD294, or any combination thereof. Further, in some aspects, the basophil identification markers can be any of: CD13, CD44, CD107a, and CD54, or any combination thereof. Additionally or alternatively, the basophil activation markers can be any of CD63, CCR3, CD123, CD63, CD203c (linage specific marker), HLA-DR-, CD300a, CD164, CD13, and CD107a, or any combination thereof. In some embodiments, a rapid activation can be associated with CD13, CD164, and Cd203a. In some embodiments, a slower activation can be associated with CD63 and CD107a.

The one or more immune cells and one or more immune cell activation markers can be T cell identification and T cell activation markers, such as CD25, CD26, CD27, CD28, CD30, CD40L, CD71, CD95, CD134, CD154, and HLA-DR, or any combination thereof. Additionally or alternatively, the one or more immune cells and one or more immune cell activation markers can be dendritic cell identification and dendritic cell activation markers, such as CD1a, CD1b, CD40, CD80, CD86, CD128b, and CD209, or any combination thereof. Further, the dendritic cell identification marker can be CD1a or CD1b. In some embodiments, CD86 is used to identify monocytes and dendritic cells. Furthermore, the dendritic cell activation marker can be including any of CD40, CD80, CD86, and CD209, or any combination thereof.

The one or more immune cells and one or more immune cell activation markers can be mast cell identification and mast cell activation markers including one of CD2, CD25, CD35, CD88, CD203a, and CD117, or any combination thereof. In some embodiments, the mast cell identification marker can be CD117. The mast cell activation marker can be any of CD2, CD25, CD35, CD88, and CD203a, or any combination thereof.

The one or more immune cells and one or more immune cell activation markers can be eosinophil identification and eosinophil activation markers, such as CD15, CD23, CD52, CD53, CD88, CD129, CD69, CD62L (decrease), CCR3, CCR9, CCR4, and CXCR3, or any combination thereof. In some embodiments, the eosinophil identification marker can be any of CD15, CD23, CD52, CD53, CD88, and CD129, or any combination thereof. Additionally or alternatively, the eosinophil activation marker can be any of of CD69, CD62L (decrease), CD52, CCR3, CCR9, CCR4, and CXCR3, or any combination thereof.

The one or more immune cells and one or more immune cell activation markers can be monocyte identification and monocyte activation markers including any of CD14, CD45, CD86, CD16, CD69, TNFα, CCR5, CD38, CX3CR1, CD11b, and HLA-DR, or any combination thereof. Further, the monocyte identification marker can be CD14 or CD45. In some embodiments, CD86 can be used to identify monocytes and dendritic cells. For example, the monocyte activation marker can be any of CD16, CD69, TNFα, CCR5, CD38, CX3CR1, and CD11b, or any combination thereof. The CD14+ cell activation marker can be any of TNF-α, CCR5, CD11b, CD69 (early activation), and HLA-DR (late activation), or any combination thereof.

In some aspects, blood can be brought into contact with detectably-labeled antibodies that specifically bind CD3, CD45, CD69, CD71, CD203c, and CD294. Further, the detection of the labeled antibodies bound to one or more of the basophils, T cells, dendritic cells, mast cells, eosinophils, and/or monocyte cells can comprise using a flow cytometer to detect the labeled antibodies.

In some embodiments, where it is determined that one or more of the basophils, T cells, dendritic cells, mast cells, eosinophils, and/or monocyte cells were activated by the one or more allergens, the patient is treated by desensitizing the patient to the one or more allergens by administering to the patient an immunotherapy regimen for reducing the patient's allergic response to the one or more allergens.

In some aspects, the procedures disclosed herein for treatment of allergy sensitivity can be repeated after a predetermined time interval. The time interval can be any suitable time interval, for example about one year to about five years. Further, for vaccine efficacy, particular T cell populations, ratios of different T cell types, as well as cytokine release, can be examined with vaccine antigen stimulation.

In some aspects, methods for treating a patient by vaccination are disclosed herein. The methods for treating the patient can include inserting a sample of blood from the patient into a capillary tube or capillary blood collection device chamber comprising sidewalls coated with one or more vaccine antigens, and allowing the blood to interact with the one or more antigens for a period of time sufficient to activate specific immune cells in the blood. During or after allowing the blood to interact with the one or more vaccine antigens, the blood can be brought into contact with a plurality of detectably-labeled antibodies that specifically bind to one or more T cell and/or B cell identification markers and/or one or more T cell and/or B cell activation markers. The specifically labeled antibodies bound to the one or more of the T cells and/or B cells can be detected to determine whether one or more of the T cells and/or B cells in the blood were activated by the vaccine antigen. The patient can be treated by vaccinating the patient with a vaccine comprising the one or more vaccine antigens or by administering a booster vaccine to the patient with a vaccine comprising the one or more vaccine antigens, if it is determined that one or more of the T cells and/or B cells were not activated by the one or more vaccine antigens.

The vaccine antigen can be a bacterial antigen. In some embodiments, the bacterial antigen can be a tuberculosis antigen.

The vaccine antigen can be a virus antigen. In some embodiments, the virus antigen can be an HPV viral antigen. Additionally or alternatively, the virus antigen can be a hepatitis virus antigen. In some embodiments, the hepatitis virus antigen can be a hepatitis A virus antigen, hepatitis B virus antigen, hepatitis C virus antigen, hepatitis D virus antigen, or hepatitis E virus antigen.

The period of time can be any suitable period of time, for example from about 1 hour to about 24 hours.

In some embodiments, the one or more T cell markers can be CD4+ T_{Reg} (regulatory T cell) cell markers including any of CD4, CD25, CD26, CD31, CD39, CD127, CD152, STAT5/FoxP3, TGF-beta, and IL-10, or any combination thereof. The one or more T cell markers are T_{CM} (central memory T cell) cell markers can be any of L-selectin, CCR7, and IL-2, or any combination thereof. The one or more T cell markers can be naive T_{SCM} (stem memory T cell) cell markers, including any of CD27, CD28, CD45R0, CD45RA, CD62L, CD95, CCR7, CXCR3, LFA-1, and IL-2Rβ13, or any combination thereof.

The one or more activated T cell markers can be any of CD25, CD44, CD62, and CD69, or any combination thereof. Additionally or alternatively, the one or more T cell markers can be Th17 cell markers, including any of CD4, TGF-beta, IL-6, STAT3/ROR-alpha, ROR-gamma T, IL-17, IL-21, and IL-22, or any combination thereof. The one or more T cell markers can be Th2 cell markers, including of CD4, IL4, STAT6/GATA-3, IL-4, IL-5, and IL-13, or any combination thereof. The one or more T cell markers can be Th1 cell markers, including any of CD4, IFN-gamma, IL-12, STAT4/T-bet, and IFN-gamma, or any combination thereof. The one or more T cell activation markers can be CD4+ T cell activation markers, including any of IL-4, IL-17 and CD40L, or any combination thereof.

The one or more T cell activation markers can be CD4+ and CD8+ T cell activation markers, including any of IFN-gamma, IL-2 and TNF-alpha, or any combination thereof. The B cell markers and B cell activation markers can be memory B cell identification and B cell activation markers including any of CD27, CD21, CD19, CD20, CD40, CD40L, CD84, CD70, CD86, CD38, MEW Class II, CXCR 4, CXCR5, and CXCR7, or any combination thereof.

The B cell markers and B cell activation markers can be B cell identification and specific memory B cell activation markers, including any of CD27, CD19, CD20, CD40, CD40L, IgA, IgG, IgD, Pex5, OBF1, and SPI-B, or any combination thereof. The B cell markers and B cell activation markers can be memory B cell identification and memory B cell activation markers, including any of CD27, CD20, CD40, and CD40L, or any combination thereof. In some embodiments, the blood can be brought into contact with detectably-labeled antibodies that specifically bind CD25, CD27, CD44, CD62, and CD69.

In some embodiments, detecting the labeled antibodies bound to one or more of the CD4+ T_{Reg} cells, T_{CM} cells, T_{SCM} cells, or memory B-cells can comprise using a flow cytometer. Additionally or alternatively, detecting the labeled antibodies bound to one or more of the CD19+, CD27+, IgD-memory B cells can comprise using a flow cytometer to detect the labeled antibodies.

In some embodiments, the methods can further comprise repeating steps a)-d) after a time interval. The time interval can be any suitable time interval, for example at least about three months, at least about six months, at least about one year, least about three years, or at least about five years.

In some embodiments, the methods can further comprise obtaining a blood sample from the patient. The blood sample can be collected in a small volume capillary tube. For example, the capillary tubes can have a volume of from about 0.5 mL to about 3 mL. Suitable volumes include, but are not limited to, about 0.5 mL, about 1 mL, about 1.5 mL, about 2 mL, about 2.5 mL, or about 3 mL. Tubes can comprise a plastic, polymer, rubber, or glass. In some embodiments, the sample of blood is a volume of 2 ml or less.

The interior side walls of the blood collection capillary tubes can be coated with the one or more allergens or vaccine antigens. In some embodiments, only one particular allergen or vaccine antigen is included per tube. The one or more antigens can comprise any antigens that activate immune responsive cells in the blood. The one or more antigens can comprise any antigens that activate in the blood. The one or more antigens or allergens can be coated onto the interior side walls of the capillary tubes according to any suitable methodology. In some embodiments, the antigens or allergens can be coated by filling the tubes with a composition of the antigens or allergens and a liquid carrier, then drying or lyophilizing the tubes to remove the liquid carrier, leaving behind the antigen or allergen. In such cases, the antigen or allergen can be re-suspended when brought into contact with the blood sample. In some embodiments, the capillary tubes can comprise an anti-coagulant agent to keep the blood in the tube in liquid form during the immune cell-antigen or -allergen interaction. In the capillary tubes, the antigens or allergen can freely interact with the blood cells.

The antigens or allergens and blood cells can interact for a period of time sufficient to allow immune cells in the blood to become activated. Generally, any suitable amount of time can be used. For example, the period of time can be from about 1 hour to about 48 hours, or from about 4 hours to about 24 hours. The interaction can be carried out in an incubator that is controlled for temperature, humidity, and other environmental conditions. In some embodiments, the interaction can be carried out at body temperature, or about 37 C.

In some embodiments, the detectable label can be a fluorescent label. In some embodiments, the antibodies do not cross-react with other allergens or vaccine antigens, such that a plurality of antibodies is used, with each member of the plurality specifically binding to a particular allergens or vaccine antigen of interest. After allowing the detectably-labeled antibodies to contact the blood and bind to their allergen or vaccine antigen, unbound antibodies can be washed from the cells. In addition, the red blood cells in the patient blood can be lysed or otherwise removed from the blood at some point prior to analysis.

Cell activation can be determined by detecting one or more of the labels on the cell-bound antibodies. Thus, for example, fluorescence microscopy or flow cytometry can be used for detection. In some embodiments, flow cytometry is used. The label detection can permit a determination of whether the cells were activated, for example, by establishing the fluorescent intensity of the markers on activated cells relative to un-activated or unstimulated cells, which can be run in parallel as appropriate controls.

In many cases, a particular patient can have multiple allergies at one time. In some embodiments, the methods can be repeated using any plurality of allergen-containing capillary tubes, with each tube containing one or more allergens from different allergenic agents. The specific cell activation signature can comprise a particular subset of cell activation markers.

The vaccine and/or allergy efficacy methods can also be repeated over time. In some embodiments, the methods can be repeated following a treatment regimen, to determine if the treatment has improved the allergy or infection. The methods can also be repeated based on encountering an individual or group in a different geographic location. The period of time between repeats is not critical, and can vary according to the needs of the individual patients, the needs of particular healthcare providers.

In some embodiments, specific patterns of cellular activation, or low levels of cell activation following vaccine antigen stimulation, can be associated with a lower immunological protection. Immunological protection can then be enhanced by the administration of booster vaccines to the patient with a vaccine comprising the vaccine antigen.

The present disclosure also provides methods for treating allergy sensitivity in a patient in need thereof, comprising: a) requesting a test providing the results of an analysis of a sample of blood from the patient for the presence of a statistically significant population of one or more immune cells activated by one or more allergens determined by contacting the blood with a plurality of detectably-labeled antibodies that specifically bind to: i) one or more basophil identification and basophil activation markers including one of CCR3, CD3, CD13, CD44, CD45, CD54, CD63, CD69, CD71, CD107a, CD107b, CD123, CD164, CD203a, CD203c, CD294, HLA-DR-, and CD300a, or any combination thereof; ii) one or more basophil identification and basophil activation markers including of CD3, CD13, CD45, CD63, CD69, CD71, CD107a, CD164, CD203c, and CD294, or any combination thereof; iii) one or more T cell identification and T cell activation markers including one of CD25, CD26, CD27, CD28, CD30, CD40L, CD71, CD95, CD134, CD154, and HLA-DR, or any combination thereof; iv) one or more dendritic cell identification and dendritic cell activation markers including one of CD1a, CD1b, CD40, CD80, CD86, CD128b, and CD209, or any combination thereof; v) one or more mast cell identification and mast cell activation markers including one of CD2, CD25, CD35, CD88, CD203a, and CD117, or any combination thereof; vi) one or more eosinophil identification and eosinophil activation markers including one of CD15, CD23, CD52, CD53, CD88, CD129, CD69+, CD62L (decrease), CCR3, CCR9, CCR4, and CXCR3, or any combination thereof; vii) one or more monocyte identification and monocyte activation markers including any of CD14, CD45, CD86, CD16, CD69, TNFα, CCR5, CD38, CX3CR1, CD11b, and HLA-DR, or any combination thereof; and/or viii) one or more immune cell markers including any of CD3, CD45, CD69, CD71, CD203c, and CD294; wherein the patient is diagnosed with an allergy sensitivity if a statistically significant population of activated basophils, T cells, dendritic cells, mast cells, eosinophils, and/or monocyte cells are detected in the blood; and b) treating the patient comprising: i) desensitizing the patient to the one or more allergens; ii) counseling the patient to avoid contact with the one or more allergens; and/or iii) administering to the patient a medicine that reduces the allergy symptoms.

The present disclosure also provides methods for treating allergy sensitivity in a patient in need thereof, comprising: a) analyzing a blood sample from the patient for the presence of a statistically significant population of immune cells activated by one or more allergens compared to blood from a healthy patient; and b) treating the patient by: i) desensitizing the patient to the one or more allergens; ii) counseling the patient to avoid contact with the one or more allergens; and/or iii) administering to the patient a medicine that reduces the allergy symptoms; wherein the presence of activated immune cells is determined by contacting the blood with a plurality of detectably-labeled antibodies that specifically bind to: i) one or more basophil identification and basophil activation markers including any of CCR3, CD3, CD13, CD44, CD45, CD54, CD63, CD69, CD71, CD107a, CD107b, CD123, CD164, CD203a, CD203c, CD294, HLA-DR-, and CD300a, or any combination thereof; ii) one or more basophil identification and basophil activation markers including any of CD3, CD13, CD45, CD63, CD69, CD71, CD107a, CD164, CD203c, and CD294, or any combination thereof; iii) one or more T cell identification and T cell activation markers including any of CD25, CD26, CD27, CD28, CD30, CD40L, CD71, CD95, CD134, CD154, and HLA-DR, or any combination thereof; iv) one or more dendritic cell identification and dendritic cell activation markers including any of CD1a, CD1b, CD40, CD80, CD86, CD128b, and CD209, or any combination thereof; v) one or more mast cell identification and mast cell activation markers including any of CD2, CD25, CD35, CD88, CD203a, and CD117, or any combination thereof; vi) one or more eosinophil identification and eosinophil activation markers including any of CD15, CD23, CD52, CD53, CD88, CD129, CD69+, CD62L (decrease), CCR3, CCR9, CCR4, and CXCR3, or any combination thereof; vii) one or more monocyte identification and monocyte activation markers including any of CD14, CD45, CD86, CD16, CD69, TNFα, CCR5, CD38, CX3CR1, CD11b, and HLA-DR, or any combination thereof; and/or viii) one or more immune cell markers including any of CD3, CD45, CD69, CD71, CD203c, and CD294.

### EXAMPLES

### Example 1: T cell Activation Detection by Flow Cytometry

Human whole blood can be obtained and diluted 1:5 in RPMI media and stimulated with 0, 20, 40, 60, 80, and 100 μg/mL (of blood) of phytohemagglutinin (PHA) for about 20 hours at 37° C. and 5% CO₂. In parallel, blood samples can be treated with a buffer (no PHA) as a stimulation control. Following the stimulation period, blood samples can be stained with fluorescently-labeled antibodies to T cell markers, including CD4, CD25, CD69, and CD71.

Fluorescently-labeled (FITC, APC, PE, PerCP-Cy5, and APC-Cy7) antibodies can be mixed with 100 μIL of PHA-stimulated blood samples or with unstimulated blood control samples in parallel, and each of the samples were can then be incubated, protected from light, for a minimum of 30 minutes. Following incubation with the labeled antibodies, the samples can be washed with BSA Stain Buffer. In parallel, samples can be separately incubated with isotype control antibodies.

Following washing, red blood cells can be lysed with 1 mL of 1× Pharm Lyse™ by incubation with this reagent for 15 minutes. Samples can be centrifuged, and washed again with BSA Stain Buffer. All samples can be fixed and permeabilized with eBiosciences Fix and Perm solutions according to the manufacturer's instructions.

FoxP3 can be added to the fixed/permeabilized samples, and the samples can be incubated for a minimum of 30 minutes. Samples can be washed with BSA Stain Buffer, and 1 μL of Violet Live/Dead is added to the samples and incubated, protected from light, for a minimum of 15 minutes.

Samples and controls can be run through a FACSAria III flow cytometer. The flow cytometer can be calibrated, and compensation can be performed according to standard requirements and procedures. Cells can be suspended at a density of 1,000,000 cells per mL in a tube, and run through the flow cytometer for analysis. One hundred thousand events can be collected per analysis.

T cells can be identified by their forward (FSC) and side (SSC) scatter profiles, and gated accordingly. T cells can further be analyzed according to the T cell marker profiles (Fox3, CD4, CD25, cD69, and CD71).

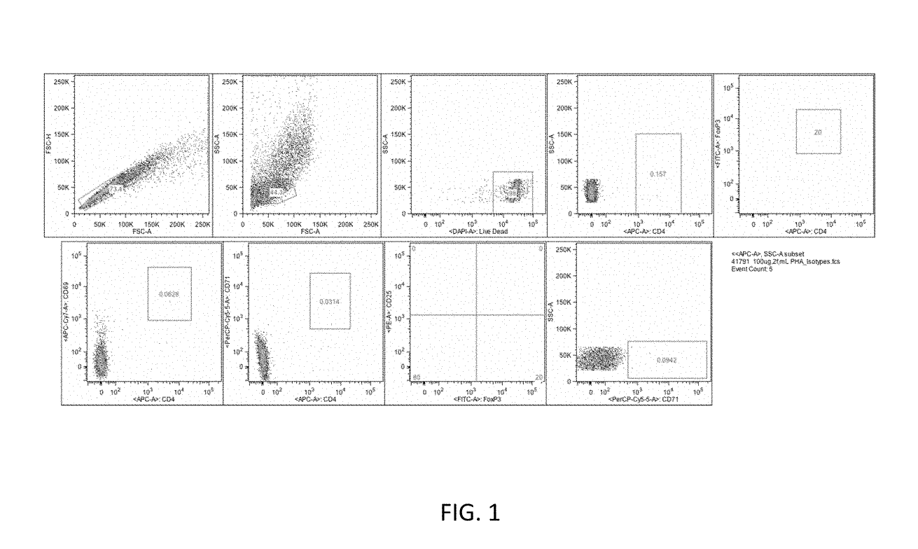

FIG. 1 depicts the results of T cell activation marker screens on non-stimulated (no PHA) blood samples. As shown, the T cells in the non-stimulated samples did not show appreciable staining for any of Fox3, CD4, CD25, cD69, and CD71.

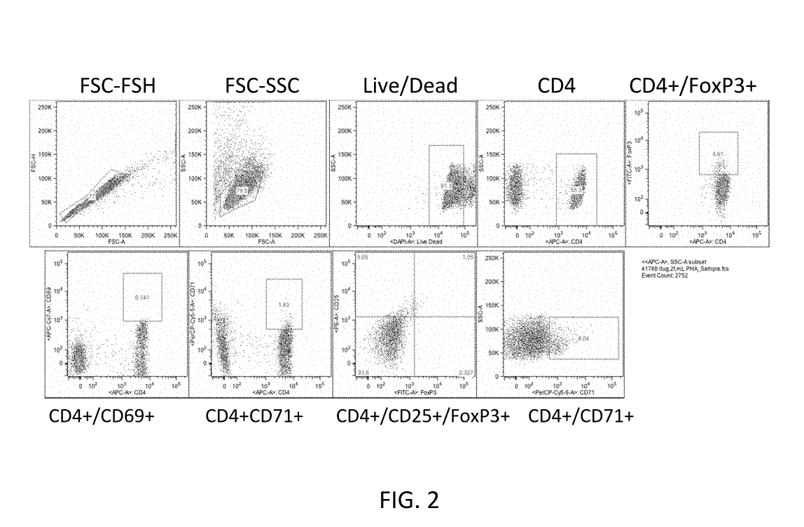

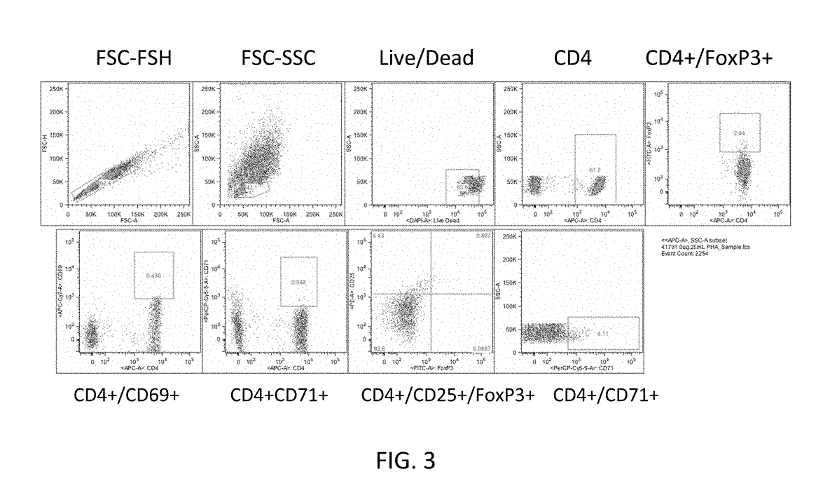

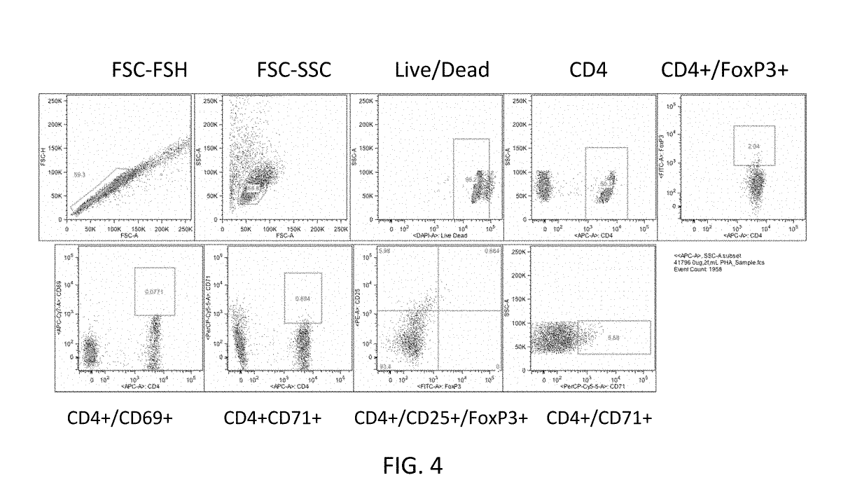

FIGS. 2, 3, and 4 illustrate the results of T cell activation marker screens on PHA-stimulated blood samples, based on increasing concentrations of PHA.

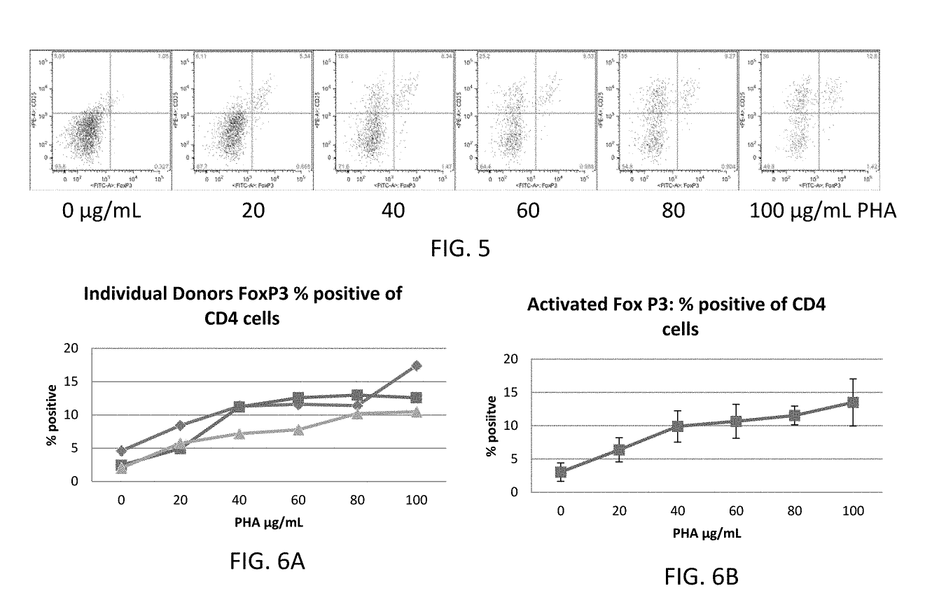

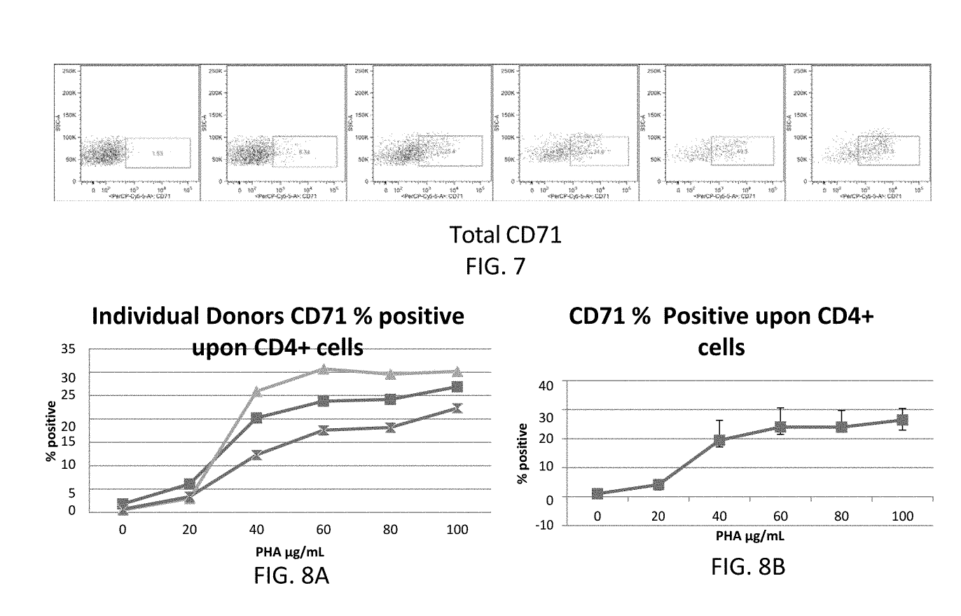

FIGS. 5, 6A, and 6B show the enhancement of the level of FoxP3 expression on CD4+ cells with increasing amounts of PHA (from 0 micrograms to 100 micrograms over 20 microgram increments). FIG. 6A graphs the percentage of FoxP3-positive CD4+ cells in select individual blood donors. FIG. 6B graphs the percentage of FoxP3-positive CD4+ cells in the pool of blood donors.

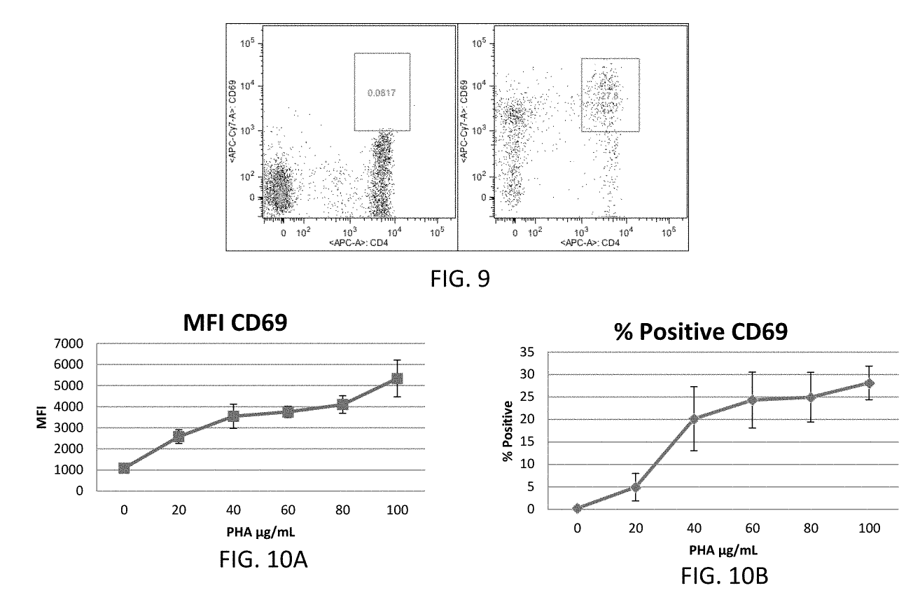

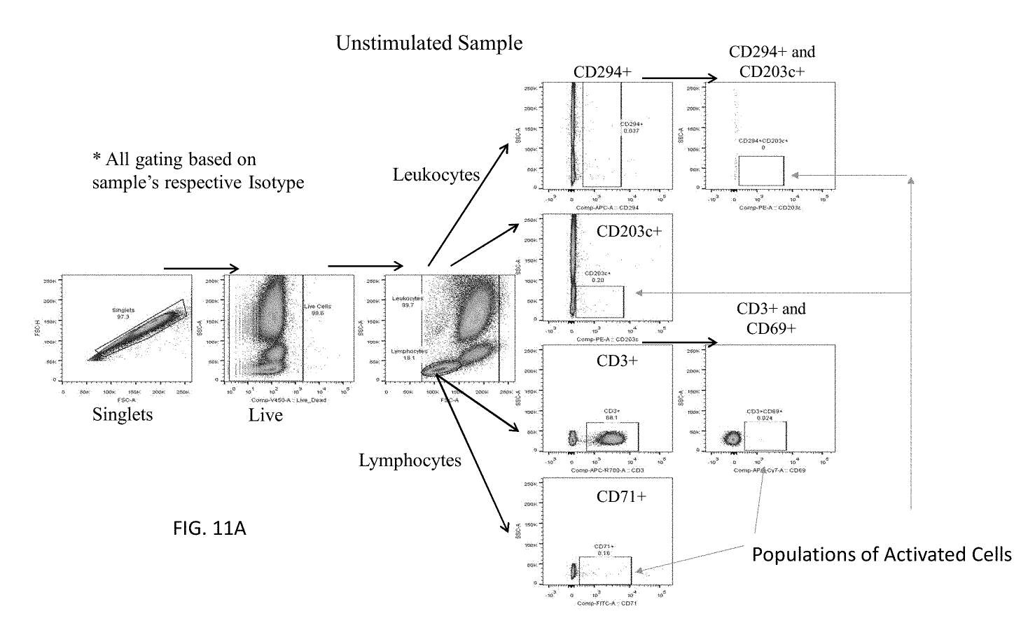

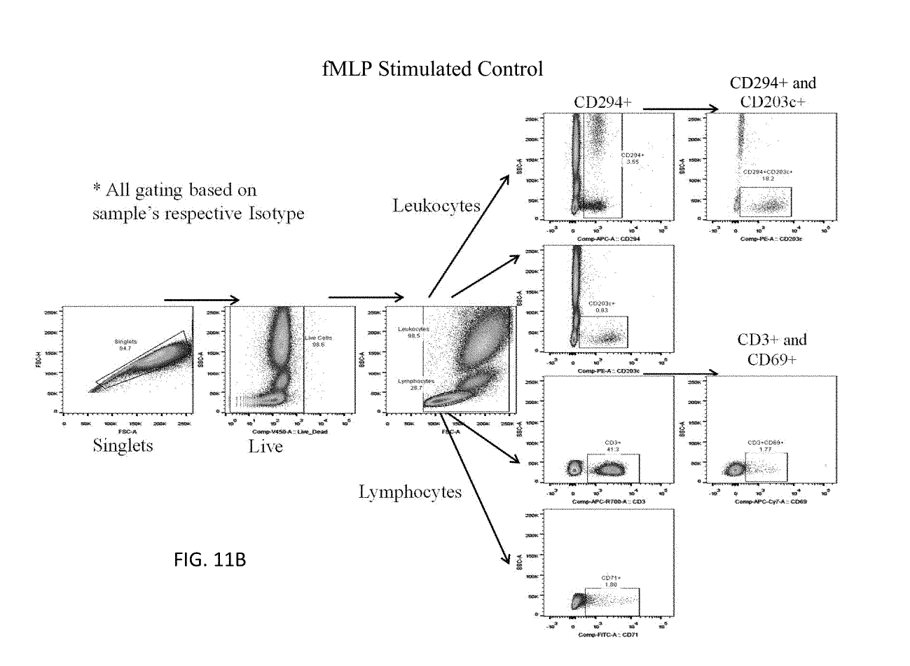

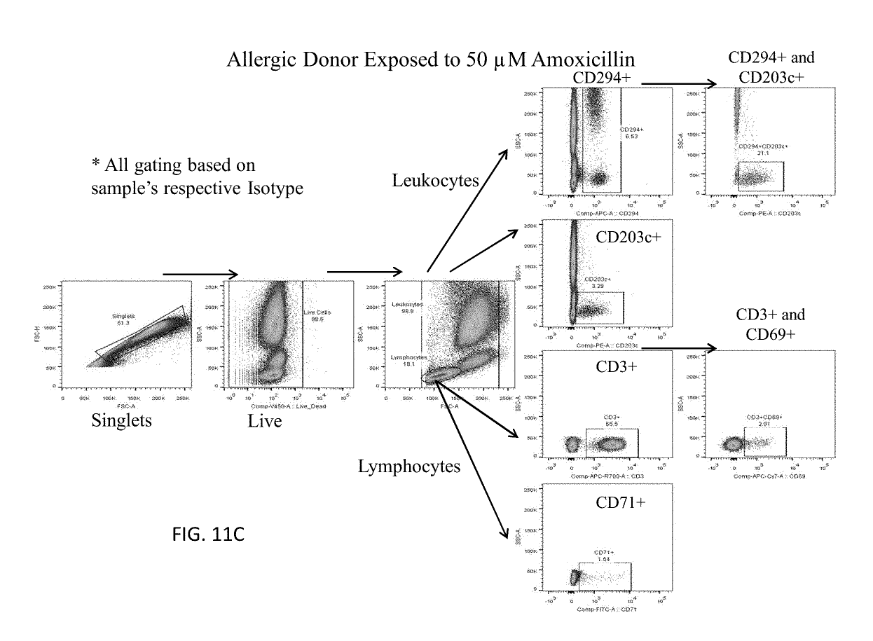

FIGS. 7, 8A, 8B, 9, 10A, and 10B include representative flow cytometry plots. Specifically, FIG. 8A and 8B illustrate results of cell stimulation with 10 μg of PHA. FIG. 9 illustrates the results of cell stimulation with 20 μg of PHA. FIGS. 10A and 10B illustrate the results of cell stimulation with 30 μg of PHA). As shown, PHA induces a substantial increase in CD4 expression on the T cells, and further induces a significant increase in the expression of CD25, CD69, CD71, and FoxP3 on the T cells.

Further, FIGS. 7, 8A, and 8B show the enhancement of the level of CD71 expression on CD4+ cells with increasing amounts of PHA (from 0 micrograms to 100 micrograms over 20 microgram increments—plots moving from left to right). FIG. 8A graphs the percentage of CD71-positive CD4+ cells in select individual blood donors. FIG. 8B graphs the percentage of CD71-positive CD4+ cells in the pool of blood donors.

FIGS. 9, 10A, and 10B show the enhancement of the level of CD69 expression on CD4+ cells with increasing amounts of PHA (from 0 micrograms (left) to 100 micrograms (right). FIG. 10A graphs the mean fluorescence intensity (MFI) of CD69 staining in the pool of blood donors over increasing amounts of PHA. FIG. 10B graphs the percentage of CD69-positive CD4+ cells in the pool of blood donors.

These data show that normal whole blood CD71, CD69, FoxP3 and CD25+ cells are stimulated by 20-100 μg/mL PHA. These experiments were repeated using a combination of CD3 and CD28 to stimulate T cells in the blood samples (PHA stimulation was run in parallel). The data showed that CD3/CD28 stimulation induced enhanced expression of CD4, CD25, CD69, CD71, and FoxP3 at levels similar to PHA stimulation (data not shown). Although the present disclosure has largely been described in connection with a flow cytometer for provided test results related to a blood sample, the present disclosure contemplates alternative analytical systems for providing such test results, for example, fluorescent microscope based systems.

### Example 2: The Detection of Leukocyte and Lymphocyte Activation Markers Involved in Allergic Response

Human whole blood was obtained from a normal control individual and from an individual with a severe allergy to Amoxicillin. The blood was diluted 1:5 in RPMI media, then stimulated with either 50 μM Amoxicillin for 20 minutes to 1 hour at 37° C. and 5% CO₂. In parallel, blood samples were treated with a buffer as a stimulation control, or with 50 μg/mL of the potent polymorphonuclear leukocyte (PMN) chemotactic factor fMLP (N-Formylmethionyl-leucyl-phenylalanine).

Following the stimulation period, blood samples were stained with fluorescently-labeled antibodies to leukocyte activation markers CD294, CD203a or lymphocyte activation markers CD71 and CD69 with the use of CD3 to identify CD3+ lymphocytes. Each of the samples were then incubated, protected from light, for a minimum of 30 minutes. Following incubation with the labeled antibodies, the samples were washed with BSA Stain Buffer. In parallel, samples were separately incubated with isotype control antibodies.

Following washing, red blood cells were lysed with 1 mL of 1× Pharm Lyse™ by incubation with this reagent for 15 minutes. Samples were centrifuged, and washed again with BSA Stain Buffer.

Samples and controls were then run through a FACS CANTO flow cytometer. The flow cytometer was calibrated, and compensation was performed according to standard requirements and procedures. Cells were suspended at a density of 1,000,000 cells per mL in a tube, and run through the flow cytometer for analysis. One hundred thousand events were collected per analysis.

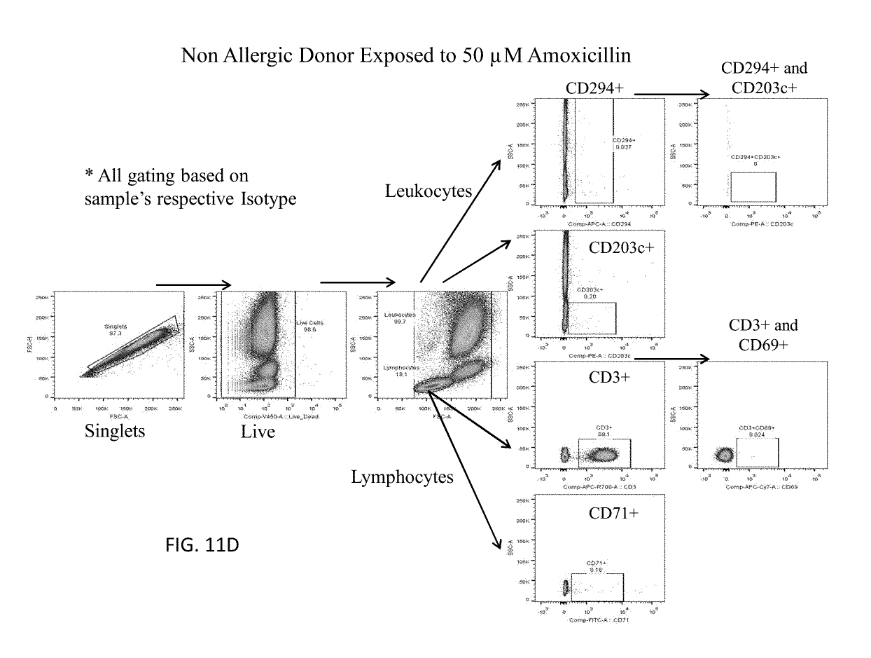

Live cells were gating based on their low staining with FVS450. Lymphocytes and leukocytes were identified by their forward (FSC) and side (SSC) scatter profiles, and gated accordingly. CD+ lymphocytes were then further analyzed according to activation markers CD69 and CD71. Representative flow cytometry plots showing these lymphocyte marker analyses are shown in FIGS. 11A, 11B, 11C, and 11D.

FIG. 11A shows the results of leukocyte and lymphocyte activation marker screens on non-stimulated (no PHA) blood samples. As shown, the lymphocyte and leukocytes in the non-stimulated samples did not show appreciable staining for any of CD294, CD203a, CD69, and CD71. Boxes identify area where a positive cell signal would be indicative of an allergic response after stimulation.

FIG. 11B shows the results of lymphocyte and leukocyte cell activation marker screens on fMLP-stimulated blood samples. As shown, fMLP induced a substantial increase in CD294 and CD203a expression on leukocytes, and further induced a significant increase in the expression of CD71 and CD69 on lymphocytes. Activated cells are identified in the boxes indicative of a positive cell mediated immune response.

FIGS. 11C and 11D shows the result of exposure from blood from a known allergic donor (highly sensitive to Amoxicillin) and a non-allergic donor. Referring to FIG. 11C, activated cells are identified in the boxes, indicative of a positive immune response to the Amoxicillin. The allergic donor demonstrated increased expression of CD294 and CD203a on leukocytes and CD69 and CD71 on CD3+ lymphocytes in response to Amoxicillin exposure. The non-allergic donor did not show an increase in CD294 and CD203a on leukocytes or CD71 or CD69 on lymphocytes, showing a biomarker profile similar to unstimulated sample control. No cell mediated immune response is evident after exposure to Amoxicillin (see, FIG. 11D).

Various terms relating to aspects of the disclosure are used throughout the specification and claims. Such terms are to be given their ordinary meaning in the art, unless otherwise indicated. Other specifically defined terms are to be construed in a manner consistent with the definitions provided herein.

As used herein, the terms “a” or “an” mean “at least one” or “one or more” unless the context clearly indicates otherwise.

As used herein, the term “about” means that the recited numerical value is approximate and small variations would not significantly affect the practice of the disclosed embodiments. Where a numerical value is used, unless indicated otherwise by the context, “about” means the numerical value can vary by ±10% and remain within the scope of the disclosed embodiments.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive and open-ended and include the options following the terms, and do not exclude additional, unrecited elements or method steps.

As used herein, the phrase “in need thereof” means that the “individual,” “subject,” or “patient” has been identified as having a need for the particular method, prevention, or treatment. In some embodiments, the identification can be by any means of diagnosis. In any of the methods, preventions, and treatments described herein, the “individual,” “subject,” or “patient” can be in need thereof. In some embodiments, the “individual,” “subject,” or “patient” is in an environment or will be traveling to an environment, or has traveled to an environment in which a particular disease, disorder, or condition is prevalent.

The terms “measure” or “determine” are used interchangeably, and refer to any suitable qualitative or quantitative determinations.

The terms “subject” or “patient” are used interchangeably. A subject can be any animal, including mammals such as companion animals, laboratory animals, and non-human primates. In some embodiments, the patient is a human.

As used herein, the terms “treat,” “treated,” or “treating” mean therapeutic treatment wherein the object is to prevent or slow down (lessen) an undesired physiological condition, disorder or disease, or obtain beneficial or desired clinical results. For purposes herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response, optionally without excessive levels of side effects. Treatment also includes prolonging survival as compared to expected survival if not receiving treatment.

It should be appreciated that particular features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable sub-combination.

Although the present disclosure has largely been described in connection with a flow cytometer for provided test results related to a blood sample, the present disclosure contemplates alternative analytical systems for providing such test results, for example, fluorescent microscope based systems.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A method comprising:
in a capillary tube comprising sidewalls coated with one or more allergens:
exposing a blood sample from a patient to the sidewalls of the capillary tube to allow interaction of the blood sample with one or more allergens;
contacting the blood sample with a plurality of labeled antibodies that specifically bind to one or more immune cells and one or more immune cell activation markers in the blood sample;
detecting the labeled antibodies bound to the one or more immune cells and immune cell activation markers in the blood sample; and
determining whether specific patterns of immune cells in the blood sample are activated by the one or more allergens.

2. The method of claim 1, wherein the blood sample comprises less than 2 milliliter of blood.

3. The method of claim 1, wherein the blood sample comprises between 0.5 milliliter to 3 milliliters of blood.

4. The method of claim 1, further comprising allowing the blood sample to interact with the one or more allergens over a predetermined period of time.

5. The method of claim 4, wherein the predetermined period of time comprises a period of time sufficient to activate specific immune cells in the blood that are responsible for an allergic response.

6. The method of claim 4, wherein the predetermined period of time comprises from about 1 hour to about 24 hours.

7. The method of claim 1, further comprising treating the patient in an event a statistically significant population of the immune cells in the blood are activated by the one or more allergens.

8. The method of claim 7, further comprising treating the patient by at least one of: desensitizing the patient to the one or more allergens; counseling the patient to avoid contact with the one or more allergens; or administering to the patient a medicine that reduces allergy symptoms caused by the one or more allergens.

9. The method of claim 8, wherein desensitizing the patient to the one or more allergens comprises administering to the patient an immunotherapy regimen for reducing the patient's allergic response to the one or more allergens.

10. The method of claim 1, further comprising detecting the specific patterns using one or more flow cytometry protocols.

11. The method of claim 1, wherein the one or more allergens comprise at least one of a food allergen, tree nut allergen, a peanut allergen, a milk allergen, an egg allergen, a shell fish allergen, a drug allergen, an antibiotic allergen, a penicillin allergen, an insect allergen, arachnid venom, bee venom, wasp venom, spider venom.

12. The method of claim 1, wherein the one or more immune cells and one or more immune cell activation markers comprise basophil identification and basophil activation markers including at least one of: CCR3, CD3, CD13, CD44, CD45, CD54, CD63, CD69, CD71, CD107a, CD107b, CD123, CD164, CD203a, CD203c, CD294, HLA-DR-, and CD300a, or a combination thereof.

13. The method of claim 1, wherein the one or more immune cells and one or more immune cell activation markers comprise T cell identification and T cell activation markers including at least one of CD25, CD26, CD27, CD28, CD30, CD40L, CD71, CD95, CD134, CD154, and HLA-DR, or a combination thereof.

14. The method of claim 1, wherein the one or more immune cells and one or more immune cell activation markers comprise dendritic cell identification and dendritic cell activation markers including at least one of CD1a, CD1b, CD40, CD80, CD86, CD128b, and CD209, or a combination thereof.

15. The method of claim 1, wherein the one or more immune cells and one or more immune cell activation markers comprise mast cell identification and mast cell activation markers including at least one of CD2, CD25, CD35, CD88, CD203a, and CD117, or a combination thereof.

16. The method of claim 1, wherein the one or more immune cells and one or more immune cell activation markers comprise eosinophil identification and eosinophil activation markers including at least one of CD15, CD23, CD52, CD53, CD88, CD129, CD69+, CD62L (decrease), CCR3, CCR9, CCR4, and CXCR3, or a combination thereof.

17. The method of claim 1, wherein the one or more immune cells and one or more immune cell activation markers comprise monocyte identification and monocyte activation markers including at least one of CD14, CD45, CD86, CD16, CD69, TNFα, CCR5, CD38, CX3CR1, CD11b, and HLA-DR, or a combination thereof.

18. The method of claim 1, further comprising contacting the blood with detectably-labeled antibodies that specifically bind CD3, CD45, CD69, CD71, CD203c, and CD294.

19. The method of claim 1, further comprising detecting the labeled antibodies bound to one or more of basophils, T cells, dendritic cells, mast cells, eosinophils, or monocyte cells using a flow cytometer.

20. A method for treating a patient by vaccination comprising:
in a capillary tube comprising sidewalls coated with one or more vaccine antigens:
exposing a blood sample from a patient to the sidewalls of the capillary tube to allow interaction of the blood sample with one or more vaccine antigens;
contacting the blood sample with a plurality of labeled antibodies that specifically bind to at least one of: one or more T cells, one or more B cells, one or more T cell activation markers, or one or more B cell activation markers in the blood sample;
detecting the labeled antibodies bound to the one or more T cells or the one or more B cells in the blood sample;
determining whether the one or more T cells or the one or more B cells in the blood sample are activated by the one or more vaccine antigens; and
in an event it is determined that one or more of the T cells or B cells are not activated by the one or more vaccine antigens, treating the patient by vaccinating the patient with a vaccine comprising the one or more vaccine antigens or by administering a booster vaccine to the patient with a vaccine comprising the one or more vaccine antigens.
